SEQUENCE LISTING (56 sequences)

SEQ ID NO: 1 - protein, 15 aa, synthetic construct
  REGION 1..15: note = Peptide RAL-M
RALQALRALQALEAL

SEQ ID NO: 2 - protein, 15 aa, synthetic construct
  REGION 1..15: note = Peptide RAL2E
RALRALRALEALEAL

SEQ ID NO: 3 - protein, 15 aa, synthetic construct
  REGION 1..15: note = Peptide RAL2Q
RALRALRALQALQAL

SEQ ID NO: 4 - protein, 15 aa, synthetic construct
  REGION 1..15: note = Peptide RALQE
RALRALRALQALEAL

SEQ ID NO: 5 - protein, 15 aa, synthetic construct
  REGION 1..15: note = Peptide RALQ2E
RALRALQALEALEAL

SEQ ID NO: 6 - protein, 17 aa, synthetic construct
  REGION 1..17: note = Peptide RAL2FQ
RALFEALQALFRALEAL

SEQ ID NO: 7 - protein, 14 aa, synthetic construct
  REGION 1..14: note = Peptide RALEQE
RALRALEALQALEA

SEQ ID NO: 8 - protein, 15 aa, synthetic construct
  REGION 1..15: note = Peptide RAL2FR
RALFEALFRALEALR

SEQ ID NO: 9 - protein, 14 aa, synthetic construct
  REGION 1..14: note = Peptide RAL2F
RALFEALFRALEAL

SEQ ID NO: 10 - protein, 13 aa, synthetic construct
  REGION 1..13: note = Peptide RALF
RALEALFRALEAL

SEQ ID NO: 11 - protein, 13 aa, synthetic construct
  REGION 1..13: note = Peptide RALF_sym
RALRALFEALEAL

SEQ ID NO: 12 - protein, 16 aa, synthetic construct
  REGION 1..16: note = Peptide RALF_Q
RALEALFRALQALEAL

SEQ ID NO: 13 - protein, 16 aa, synthetic construct
  REGION 1..16: note = Peptide RALFQ_W
RALEALWRALQALEAL

SEQ ID NO: 14 - protein, 13 aa, synthetic construct
  REGION 1..13: note = Peptide RALF_W
RALEALWRALEAL

SEQ ID NO: 15 - protein, 16 aa, synthetic construct
  REGION 1..16: note = Peptide RALA4Q
RALARALARALAQALA

SEQ ID NO: 16 - protein, 15 aa, synthetic construct
  REGION 1..15: note = Peptide P8_S2
RSIVTFSLRQNAQLA

SEQ ID NO: 17 - protein, 15 aa, synthetic construct
  REGION 1..15: note = PeptideP8_S8
RSIVTFSLRQNSEQA

SEQ ID NO: 18 - protein, 12 aa, synthetic construct
  REGION 1..12: note = Peptide BA1-A06
GLHTSATNLYLH

SEQ ID NO: 19 - protein, 12 aa, synthetic construct
  REGION 1..12: note = Peptide D2
QHSIRLLTIKKP

SEQ ID NO: 20 - protein, 12 aa, synthetic construct
  REGION 1..12: note = Peptide D4
QQSIRIMTIKHP

SEQ ID NO: 21 - protein, 12 aa, synthetic construct
  REGION 1..12: note = Peptide P3
WRHPRLRCGNLL

SEQ ID NO: 22 - protein, 12 aa, synthetic construct
  REGION 1..12: note = Peptide P14
QKSRNRMTRTHP

SEQ ID NO: 23 - protein, 12 aa, synthetic construct
  REGION 1..12: note = Peptide P6
SRARLFVVTYHK

SEQ ID NO: 24 - protein, 12 aa, synthetic construct
  REGION 1..12: note = Peptide P7
HMISTMNAASRR

SEQ ID NO: 25 - protein, 12 aa, synthetic construct
  REGION 1..12: note = Peptide P8
RSIVTFSLRQNR

SEQ ID NO: 26 - protein, 12 aa, synthetic construct
  REGION 1..12: note = Peptide P13
RNTIRIRTIKHP

SEQ ID NO: 27 - protein, 12 aa, synthetic construct
  REGION 1..12: note = Peptide P15
RHSSTLRYRPLP

SEQ ID NO: 28 - protein, 16 aa, synthetic construct
  REGION 1..16: note = Peptide RAL-M-C
RALQALRALQALEALC

SEQ ID NO: 29 - protein, 16 aa, synthetic construct
  REGION 1..16: note = Peptide RAL2E-C
RALRALRALEALEALC

SEQ ID NO: 30 - protein, 16 aa, synthetic construct
  REGION 1..16: note = Peptide RAL2Q-C
RALRALRALQALQALC

SEQ ID NO: 31 - protein, 16 aa, synthetic construct
  REGION 1..16: note = Peptide RALQE-C
RALRALRALQALEALC

SEQ ID NO: 32 - protein, 16 aa, synthetic construct
  REGION 1..16: note = Peptide RALQ2E-C
RALRALQALEALEALC

SEQ ID NO: 33 - protein, 18 aa, synthetic construct
  REGION 1..18: note = Peptide RAL2FQ-C
RALFEALQALFRALEALC

SEQ ID NO: 34 - protein, 15 aa, synthetic construct
  REGION 1..15: note = Peptide RALEQE-C
RALRALEALQALEAC

SEQ ID NO: 35 - protein, 16 aa, synthetic construct
  REGION 1..16: note = Peptide RAL2FR-C
RALFEALFRALEALRC

SEQ ID NO: 36 - protein, 15 aa, synthetic construct
  REGION 1..15: note = Peptide RAL2F-C
RALFEALFRALEALC

SEQ ID NO: 37 - protein, 14 aa, synthetic construct
  REGION 1..14: note = Peptide RALF-C
RALEALFRALEALC

SEQ ID NO: 38 - protein, 14 aa, synthetic construct
  REGION 1..14: note = Peptide RALF_sym
RALRALFEALEALC

SEQ ID NO: 39 - protein, 17 aa, synthetic construct
  REGION 1..17: note = Peptide RALF_Q-C
RALEALFRALQALEALC

SEQ ID NO: 40 - protein, 17 aa, synthetic construct
  REGION 1..17: note = Peptide RALFQ_W-C
RALEALWRALQALEALC

SEQ ID NO: 41 - protein, 14 aa, synthetic construct
  REGION 1..14: note = Peptide RALF_W-C
RALEALWRALEALC

SEQ ID NO: 42 - protein, 17 aa, synthetic construct
  REGION 1..17: note = Peptide RALA4Q-C
RALARALARALAQALAC

SEQ ID NO: 43 - protein, 16 aa, synthetic construct
  REGION 1..16: note = Peptide P8_S2-C
RSIVTFSLRQNAQLAC

SEQ ID NO: 44 - protein, 16 aa, synthetic construct
  REGION 1..16: note = Peptide P8_S8-C
RSIVTFSLRQNSEQAC

SEQ ID NO: 45 - protein, 13 aa, synthetic construct
  REGION 1..13: note = Peptide BA1-A06-C
GLHTSATNLYLHC

SEQ ID NO: 46 - protein, 13 aa, synthetic construct
  REGION 1..13: note = Peptide D2-C
QHSIRLLTIKKPC

SEQ ID NO: 47 - protein, 13 aa, synthetic construct
  REGION 1..13: note = Peptide D4-C
QQSIRIMTIKHPC

SEQ ID NO: 48 - protein, 13 aa, synthetic construct
  REGION 1..13: note = Peptide P3-C
WRHPRLRCGNLLC

SEQ ID NO: 49 - protein, 13 aa, synthetic construct
  REGION 1..13: note = Peptide P14-C
QKSRNRMTRTHPC

SEQ ID NO: 50 - protein, 13 aa, synthetic construct
  REGION 1..13: note = Peptide P6-C
SRARLFVVTYHKC

SEQ ID NO: 51 - protein, 13 aa, synthetic construct
  REGION 1..13: note = Peptide P7-C
HMISTMNAASRRC

SEQ ID NO: 52 - protein, 13 aa, synthetic construct
  REGION 1..13: note = Peptide P8-C
RSIVTFSLRQNRC

SEQ ID NO: 53 - protein, 13 aa, synthetic construct
  REGION 1..13: note = Peptide P13-C
RNTIRIRTIKHPC

SEQ ID NO: 54 - protein, 13 aa, synthetic construct
  REGION 1..13: note = Peptide P15-C
RHSSTLRYRPLPC

SEQ ID NO: 55 - protein, 12 aa, synthetic construct
  REGION 1..12: note = Peptide AI-S1
VPSSGPQDTRTT

SEQ ID NO: 56 - protein, 12 aa, synthetic construct
  REGION 1..12: note = Peptide MS-S1
ATIHDAFYSAPE

(19) United States
(12) Patent Application Publication
(10) Pub. No.: US 2024/0376357 A1
(43) Pub. Date: Nov. 14, 2024

(54) Substrate-specific adhesive peptides

(71) Applicant: HENKEL AG & CO. KGAA, Düsseldorf (DE)

(72) Inventors: Iva ANIC, Düsseldorf (DE); Christian KASTNER, Düsseldorf (DE); Irmgard SCHMIDT, Solingen (DE); Roland BREVES, Mettmann (DE); Andreas TADEN, Düsseldorf (DE); Kenji ITO, Düsseldorf (DE); Shohana ISLAM, Düsseldorf (DE); Christian DEGERING, Erkrath (DE); Susanne WIELAND, Zons/Dormagen (DE)

(21) Appl. No.: 18/564,739

(22) PCT Filed: Dec. 7, 2022
(86) PCT No.: PCT/EP2022/084762

§ 371 Date: Nov. 28, 2023

(30) Foreign Application Priority Data
Dec. 16, 2021 (DE) 10 2021 133 427.6

(51) Int. Cl. C09J 189/00; C07K 7/06; C07K 7/08; C07K 14/00

(52) U.S. Cl. CPC C09J 189/00; C07K 7/06; C07K 7/08; C07K 14/00; C09J 2489/00

(57) ABSTRACT

The invention relates to a peptide as described herein. The invention further relates to a peptide or polypeptide which comprises at least two of the peptides according to the invention, to a nucleic acid sequence encoding the peptide according to the invention, to a means comprising at least one peptide according to the invention and to the use of peptides of this kind for adhesion to plastic surfaces.

The invention relates to a peptide as described herein. Furthermore, the invention relates to peptides or polypeptides which comprise at least two of the peptides described herein in the form of multimers, to a nucleic acid sequence encoding the peptide according to the invention, to a means comprising at least one peptide according to the invention and to the use of such peptides or means for binding/adhesion to plastic surfaces.

The development of adhesive peptides is the subject of intensive research. In particular, peptides that specifically bind to or interact with oxidic surfaces such as metal surfaces have already been described (WO 2014/072313 A1). Adhesive peptides are, however, also of interest for many other surfaces which, due to their material or their surface properties, make it difficult, for example, to easily bond or attach other objects, substances or compounds. In the case of such surfaces, those with low surface energies, such as those often found in certain plastics, are particularly problematic. Such plastics are also referred to in the prior art as low surface energy polymers (LSEPs) and comprise, for example, many polyolefins.

The inventors have found and developed specific peptides which, surprisingly, are suitable for adhesion to plastic surfaces, in particular LSEPs (low surface energy polymer) surfaces, and partially or completely solve the above-mentioned problems.

Therefore, in a first aspect, the invention relates to a peptide comprising or consisting of an amino acid sequence of from 4 to 50 amino acids, where
(a) the amino acid sequence in N- to C-terminal orientation has the following sequence

(C)ₘX₁X₂X₃(X₄)ₙX₅(C)ₒ,

where
X₁ is a positively charged amino acid,
X₂ and X₃ are uncharged amino acids,
each X₄, independently of one another, is any amino acid,
X₅ is any positively charged or uncharged amino acid,
m and o are 0 or 1, where m+o=0 or 1; and
x is an integer from 0 to 46;
or
b) the amino acid sequence has at least 80% sequence identity to one of the amino acid sequences specified in SEQ ID NOs: 16-17 or 18-22.

In a further aspect, the invention relates to a multimer of the above-mentioned peptides comprising two or more of the amino acid sequences mentioned, preferably in the form of a peptide or polypeptide.

In a further aspect, the invention relates to a nucleic acid sequence encoding the peptides and polypeptides described herein.

Furthermore, the invention relates to a means comprising one or more of the peptides or polypeptides according to the invention.

Finally, the invention relates to the use of at least one peptide, or a means containing said peptide contains, for adhesion or binding to plastic surfaces, where the peptide has or consists of the amino acid sequence according to one of SEQ ID NOs: 1-27, preferably SEQ ID NOs: 1-15, 16-17 or 18-22, or is a variant thereof having at least 80%, preferably at least 90%, sequence identity to one of the specified sequences.

These and other aspects, features and advantages of the invention will become apparent to a person skilled in the art through the study of the following detailed description and claims. Any feature from one aspect of the invention can be used in any other aspect of the invention. For example, features or embodiments described for the peptide according to the invention are also to be used for the polypeptide according to the invention, the means according to the invention, the nucleic acid sequence according to the invention, or the use according to the invention, and vice versa. Furthermore, it will readily be understood that the examples contained herein are intended to describe and illustrate but not to limit the invention and that, in particular, the invention is not limited to these examples.

Unless otherwise stated, all percentages are percentages by weight (wt. %), each based on the total weight of the corresponding composition or the corresponding means. Numerical ranges which are indicated in the format “from x to y” also include the stated values. If several preferred numerical ranges are indicated in this format, it is readily understood that all ranges which result from the combination of the various endpoints are also included.

“At least one”, as used herein, refers to 1 or more, for example 1, 2, 3, 4, 5, 6, 7, 8, 9 or more. In relation to an ingredient, the specification refers to the type of ingredient and not to the absolute number of molecules. “At least one peptide” thus means, for example, at least one type of peptide; that is to say that one type of peptide or a mixture of a plurality of different peptides may be meant. Together with weight specifications, the specification relates to all compounds of the type indicated that are contained in, for example, a composition or a means; that is to say that the composition or the means does not contain any other compounds of this type beyond the specified amount of the corresponding compounds.

Numerical values, specified herein without decimal places, refer in each case to the full specified value with one decimal place. For example, “99%” stands for “99.0%.”

The invention relates to a peptide comprising or consisting of an amino acid sequence of 4 to 50 amino acids in length, preferably of at least 8, 9, 10, 11 or 12 amino acids in length. Preferred lengths are up to 40, up to 35, up to 30, or up to 25 or up to 24 amino acids. For example, the peptide may have a length of 11, 12, 13, 14, 15, 16, 17, 18, 19, 20, 21, 22, 23 or 24 amino acids, in particular 12 to 18 amino acids.

The peptides of the invention have the following amino acid sequence in N- to C-terminal orientation

(C)ₘX₁X₂X₃(X₄)ₙX₅(C)ₒ,

where
X₁ is a positively charged amino acid, preferably R or K, more preferably R,
X₂ and X₃ are uncharged amino acids, preferably selected from A, L, M, S, I and Q, more preferably from A, L, M and Q, in particular from A and L,
each X₄ independently of one another is any amino acid, preferably with the exception of P, more preferably with the exception of P and G;
X₅ is any positively charged or uncharged amino acid, preferably R or an uncharged amino acid, more preferably Q, A or L, in particular A or L,
m and o are 0 or 1, where m+o=0 or 1; and
n is an integer from 0 to 46, preferably from 6 to 20, for example 9, 10, 11, 12 or 13.

Alternatively or additionally, the peptides comprise or consist of an amino acid sequence having at least 80%, preferably at least 81%, at least 82%, at least 83%, at least 84% or at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to one of the amino acid sequences specified in SEQ ID NOs: 16-17 or 18-22.

A “peptide” in the context of the present invention is to be understood as meaning a polymer composed of amino acids, preferably the 20 proteinogenic L-amino acids, preferably of a linear structure and having up to 100 amino acids which are linked to one another via peptide bonds. According to the invention, the peptides of the invention have an amino acid sequence of 4 to 50 amino acids. In the context of the present invention, amino acids are indicated in single-letter code, where, for example, C is cysteine, R is arginine, A is alanine, and L is leucine. In particular, “C” in the above sequence (C)ₘX₁X₂X₃(X₄)ₙX₅(C)ₒ represents a cysteine residue. It is further understood that unless otherwise indicated, the amino acids in an amino acid sequence disclosed herein are linked via peptide bonds and the sequence is set forth in N- to C-terminal orientation unless otherwise indicated.

In various embodiments, the peptides according to the invention may have been chemically synthesized and/or recombinantly produced by means of protein design. Nowadays, short peptides can simply be prepared synthetically, for example via solid phase synthesis. By contrast, longer peptides and polypeptides are frequently also produced recombinantly in a host organism.

In the context of the present invention, the term “N-terminus” or “N-terminal” describes the end of the amino acid chain of the peptide according to the invention which has a free amino group.

In the context of the present invention, the term “C-terminus” or “C-terminal” describes the end of the amino acid chain of the peptide according to the invention which has a free carboxyl group.

In the context of this invention, the term “in N- to C-terminal orientation” refers to an amino acid sequence in which the sequence of amino acids is described starting from the N-terminus towards the C-terminus.

Typical acidic or negatively charged amino acids (depending on pH) are D and E.

Positively charged or basic amino acids (depending on pH) typically include R, K, and H.

Amino acids, such as G, A, C, I, L, M, F, V, P, S, T, W, Y, N and Q, are typically uncharged, i.e., neutral amino acids.

When reference is made herein to “any” amino acid, this usually means one of the 20 naturally occurring proteinogenic amino acids, i.e., one of glycine (G), alanine (A), valine (V), leucine (L), isoleucine (I), phenylalanine (F), serine (S), threonine (T), proline (P), methionine (M), cysteine (C), histidine (H), lysine (K), arginine (R), glutamine (Q), asparagine (N), aspartic acid (D), glutamic acid (E), tyrosine (Y) and tryptophan (W). Unless otherwise specified, the amino acids are typically L-amino acids. In alternative embodiments, the peptide may also consist of D-amino acids, although it may be preferred that, within the peptides described herein, D- and L-amino acids are not simultaneously present. In various embodiments, any such amino acid encompasses all of the aforementioned amino acids with the exception of proline, or in some embodiments, also with the exception of proline and glycine. In certain embodiments, these two amino acids are not preferred since they have helix-breaking properties and may therefore adversely affect the secondary structure of the peptides.

In preferred embodiments, the peptide according to the invention has a total charge of −2 to +12, preferably of 0 to +8, more preferably of 0 to +4, in particular of 0 to +2. The total charge of the peptide is based on the number of positively and negatively charged amino acids in the peptide, in particular arginine (R), lysine (K), histidine (H), aspartic acid (D) and glutamic acid (E), and results from the sum of the negative and positive charges, where in each case a positive and a negative charge cancel each other out. Thus, a peptide with 2 arginine residues and 1 glutamic acid residue would have a total charge of +1. The total charge of the peptide is preferably −2 to +12, more preferably 0 to +8, even more preferably 0 to +4, in particular 0 to +2.

In preferred embodiments, the peptide according to the invention has
(i) a total charge of from −2 to +12, preferably from 0 to +8, more preferably from 0 to +4, in particular from 0 to +2; and/or
(ii) at the N-terminus comprising the first 3-4 amino acids, a net positive charge, and/or
(iii) at the C-terminus comprising the last 3-6 amino acids, a net negative or neutral charge, preferably at least one negatively charged amino acid, in particular E; and/or
(iv) no P and even more preferably also no G and in particular no Y.

All aforementioned features, in particular (i)-(iv), may be realized individually or in any combination.

The feature that the peptide at the “N-terminus comprising the first 3-4 amino acids has a net positive charge” means that the N-terminal 3-4 amino acids comprise more positively charged amino acids than negatively charged amino acids. In various embodiments, this feature is fulfilled, for example, when the N-terminal 3-4 amino acids have 1 or 2 positively charged amino acids, i.e., H, K or R, preferably K or R, more preferably R, and no negatively charged amino acids, such as E or D. If the N-terminus contains a negatively charged amino acid, the number of positively charged amino acids must be at least 2 in order for the net charge to remain positive.

For the feature that the peptide at the “C-terminus of the last 3-6 amino acids has a net negative or neutral charge” means that the number of charged amino acids must be 0 or the number of negatively amino acids, i.e., D and E, must be greater than the number of positively charged ones. An example of such a C-terminal sequence would be, for example, EAL or the double sequence of this motif.

In various preferred embodiments, the sequence is X₁X₂X₃ RAL, RSI or RLA, preferably RAL or RLA, in particular RAL. The N-terminal sequence RAL or RLA not only advantageously has a net positive charge, it also comprises amino acids with a particularly high alpha-helix-forming potential, as will be explained further below. In some embodiments, the arginine residue may also be replaced by lysine, but the N-terminal arginine residue is particularly preferred.

It is further preferred that
(i) the sequence (X₄)ₙX₅ comprises at least one sequence X₆X₇X₈, where X₆ is a charged or uncharged amino acid, preferably R, K, E, L, A or Q, more preferably R, K, E or Q, and X₇ and X₈ independently of one another are uncharged amino acids with the exception of P and G, preferably A, L, Q or M, more preferably A or L; and/or
(ii) (X₄)ₙ comprises at least one aromatic amino acid, preferably W or F.

If the sequence X₆X₇X₈ comprises an X₆ which is R or K, the sequence X₆X₇X₈ is preferably not at the C-terminal and preferably not within the 6 C-terminal amino acids. In such cases, the sequence (X₄)ₙX₅ may comprise one or more further sequences X₆X₇X₈, which are C-terminal to the sequence, which comprise as X₆ a positively charged amino acid, with these further sequences then preferably not having a positively charged amino acid as X₆.

It is preferred that one of the sequences X₆X₇X₈, which are close to, i.e., within the 6 C-terminal amino acids, or at the C-terminus, have as X₆ a negatively charged amino acid, for example E.

If, in some embodiments, the peptide contains an aromatic amino acid selected from W and F, there is a positively or negatively charged amino acid adjacent to, in particular C-terminal, of such an aromatic amino acid; in particular there is no further aromatic amino acid adjacent to the aromatic amino acid.

The aromatic amino acids phenylalanine (F) and tryptophan (W) are preferably used as helix formers and/or for pi-stacking in the peptide sequence according to the invention. In various embodiments the aromatic amino acid tyrosine (Y) is not used in the peptide sequence since it has helix-breaking properties. In various embodiments, the peptide is therefore free of Y residues.

In the context of the present invention, “Pi-stacking” refers to the non-covalent interaction between aromatic ring systems.

Preferably,
(i) (X₄)ₙ comprises at least one sequence X₆X₇X₈, where X₆X₇X₈ is RAL or RLA, preferably RAL, and where said sequence is preferably localized in the N-terminal amino acids of positions 4-7, or at least 6-7 amino acids away from the C-terminus; and/or
(ii) (X₄)ₙX₅ comprises at least one sequence X₆X₇X₈, where X₆X₇X₈ is EAL or ELA, preferably EAL, and where said sequence is preferably not localized in the N-terminal amino acids of positions 1-6; and/or
(iii) (X₄)ₙX₅ comprises at least one sequence X₆X₇X₈, where X₆X₇X₈ is QAL or QLA, preferably QAL.

In other possible embodiments (X₄)ₙX₅ comprises at least one sequence X₆X₇X₈, where X₆X₇X₈ is QLA or EQA, where said sequence is preferably not localized in the N-terminal amino acids of positions 1-6 or 1-11.

It is further possible that (X₄)ₙX₅ comprises at least one sequence X₆X₇X₈X₉, where X₆X₇X₈X₉ is AQLA or SEQA, where said sequence is preferably not localized in the N-terminal amino acids of positions 1-6 or 1-11.

In preferred embodiments, the peptide comprises the sequence X₁X₂X₃, where X₁X₂X₃ is RAL, and (X₄)ₙX₅ comprises at least one of QAL and EAL, preferably both. In such embodiments, the peptide has the sequence

(C)ₘRAL(X₁₀)_{q}QAL(X₁₁)ᵣEAL(X₁₂)ₛ(C)ₒ, or

(C)ₘRAL(X₁₀)_{q}EAL(X₁₁)ᵣQAL(X₁₂)ₛ(C)ₒ,

where X₁₀ and X₁₁ independently of one another are any amino acids, preferably with the exception of P, more preferably with the exception of P and G;
X₁₂ is any uncharged amino acid, preferably Q, A or L, in particular A or L;
q and r are 0 or integers of 1-10, preferably 0, 1, 2 or 3; and
s is 0 or 1,
where q+r+s=0-21, preferably 0-15 or 0-9 or 1-15 or 1-9 or 1-6 or 0-6 or 0-3 or 1-3.

Furthermore, it is preferred that the peptide additionally comprises at least one further (second) sequence RAL. Said sequence may, in various embodiments, directly C-terminally follow the first RAL sequence or be separated from it by 1-3 amino acids, for example by 1 or 3 three amino acids.

In preferred embodiments, the peptide contains two sequences RAL and at least one sequence each of EAL and QAL. Preferred sequences are:

RALRAL(X₁₀)_{q}QAL(X₁₁)ᵣEAL(X₁₂)ₛ,

RALRAL(X₁₀)_{q}EAL(X₁₁)ᵣQAL(X₁₂)ₛ

RAL(X₁₀)_{q}RALQAL(X₁₁)ᵣEAL(X₁₂)ₛ,

RAL(X₁₀)_{q}RALEAL(X₁₁)ᵣQAL(X₁₂)ₛ

RAL(X₁₀)_{q}QALRAL(X₁₁)ᵣEAL(X₁₂)ₛ,

RAL(X₁₀)_{q}EALRAL(X₁₁)ᵣQAL(X₁₂)ₛ,

where X₁₀ and X₁₁ independently of one another are any amino acids, preferably with the exception of P, more preferably with the exception of P and G, for example W or For further QAL or EAL motifs;
X₁₂ is any uncharged amino acid, preferably Q, A or L, in particular A or L;
q and r are 0 or integers of 1-6, preferably 0, 1, 2, or 3; and
s is 0 or 1, preferably 0,
where q+r=0-9, preferably 0-6 or 0-3 or 1-9 or 1-6 or 1-3.

Furthermore, it is preferred in various embodiments that the peptide contains at least one W or F, preferably exactly one W or F.

Preferably, the peptide according to the invention comprises amino acids having a high alpha-helix-forming potential, with said amino acids being selected from E, A, L, M, Q, K, R, F, I, H, W and D, more preferably E, A, L, M, Q, K, R, F, I and H; even more preferably E, A, L, M, Q, K, R and F.

In preferred embodiments, the peptide consists of at least 60%, preferably at least 65%, more preferably at least 70%, in particular at least 75% or at least 80% or at least 85% or at least 90% or at least 95% of amino acids having a high alpha-helix-forming potential, with said amino acids being preferably selected from E, A, L, M, Q, K, R, F, I, H, W and D, more preferably E, A, L, M, Q, K, R, F, I and H; even more preferably E, A, L, M, Q, K, R and F.

Particularly preferably, the peptide according to the invention forms a helical secondary structure, in particular an α-helix structure with an α-helix content of at least 80%, preferably at least 85%, more preferably at least 90%, even more preferably at least 95%, in particular higher than 95%. The use of the AL or LA motif in the amino acid sequence of the peptide according to the invention can contribute to the stability of the helix structure because these amino acids have a high α-helix potential.

In preferred embodiments, the peptide has the amino acid sequence according to one of SEQ ID NOs: 1-15 or 16-17, in particular 1-15, and variants thereof, which have at least 80%, preferably at least 81%, at least 82%, at least 83%, at least 84% or at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to the specified sequence, wherein preferably the RAL motif, and more preferably also the EAL and/or QAL motif, if present, are invariable. If the RAL, EAL and QAL motifs are present in the peptide, they are preferably invariable in all the aforementioned variants.

In various embodiments, the peptide may have a high proportion of hydrophobic amino acids selected from A, L, F, W, V, M, I and P, in particular A, L, F, W, V, M and I.

Preferably, the peptide has an amino acid sequence which has a length of 10 to 24 amino acids, for example 11, 12, 13, 14, 15, 16, 17, 18, 19, 20, 21, 22, 23 or 24 amino acids, in particular 12 to 18 amino acids.

In some embodiments, the peptide according to the invention has the amino acid cysteine (C) at the C-terminus. In other embodiments, the peptide according to the invention has the amino acid cysteine at the N-terminus. This amino acid can enable coupling to other molecules, structures or substrates via the free sulfhydryl group. This amino acid therefore serves as a linking point but is typically not involved in the desired adhesive effect.

Preferably, the peptide according to the invention comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences: RALQALRALQALEAL (SEQ ID NO: 1), RALRALRALEALEAL (SEQ ID NO: 2), RALRALRALQALQAL (SEQ ID NO: 3), RALRALRALQALEAL (SEQ ID NO: 4), RALRALQALEALEAL (SEQ ID NO: 5), RALFEALQALFRALEAL (SEQ ID NO: 6), RALRALEALQALEA (SEQ ID NO: 7), RALFEALFRALEALR (SEQ ID NO: 8), RALFEALFRALEAL (SEQ ID NO: 9), RALEALFRALEAL (SEQ ID NO: 10), RALRALFEALEAL (SEQ ID NO: 11), RALEALFRALQALEAL (SEQ ID NO: 12), RALEALWRALQALEAL (SEQ ID NO: 13), RALEALWRALEAL (SEQ ID NO: 14), RALARALARALAQALA (SEQ ID NO: 15), RSIVTFSLRQNAQLA (SEQ ID NO: 16) or RSIVTFSLRQNSEQA (SEQ ID NO: 17), preferably 1-15.

In various other embodiments, the peptide according to the invention comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences: RSIVTFSLRQNAQLA (SEQ ID NO: 16), RSIVTFSLRQNSEQA (SEQ ID NO: 17), GLHTSATNLYLH (SEQ ID NO: 18), QHSIRLLTIKKP (SEQ ID NO: 19), QQSIRIMTIKHP (SEQ ID NO: 20), WRHPRLRCGNLL (SEQ ID NO:21) or QKSRNRMTRTHP (SEQ ID NO: 22), preferably 16 or 17.

In various other embodiments, the peptide according to the invention comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences: GLHTSATNLYLH (SEQ ID NO: 18), QHSIRLLTIKKP (SEQ ID NO: 19), QQSIRIMTIKHP (SEQ ID NO: 20), WRHPRLRCGNLL (SEQ ID NO:21) or QKSRNRMTRTHP (SEQ ID NO: 22).

Preferably, the peptide according to the invention comprises or consists of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the following amino acid sequences: RALQALRALQALEAL (SEQ ID NO: 1), RALRALRALEALEAL (SEQ ID NO: 2), RALRALRALQALQAL (SEQ ID NO: 3), RALRALRALQALEAL (SEQ ID NO: 4), RALRALQALEALEAL (SEQ ID NO: 5), RALFEALQALFRALEAL (SEQ ID NO: 6), RALRALEALQALEA (SEQ ID NO: 7), RALFEALFRALEALR (SEQ ID NO: 8), RALFEALFRALEAL (SEQ ID NO: 9), RALEALFRALEAL (SEQ ID NO: 10), RALRALFEALEAL (SEQ ID NO: 11), RALEALFRALQALEAL (SEQ ID NO: 12), RALEALWRALQALEAL (SEQ ID NO: 13), RALEALWRALEAL (SEQ ID NO: 14), RALARALARALAQALA (SEQ ID NO: 15), RSIVTFSLRQNAQLA (SEQ ID NO: 16), RSIVTFSLRQNSEQA (SEQ ID NO: 17), GLHTSATNLYLH (SEQ ID NO: 18), QHSIRLLTIKKP (SEQ ID NO: 19), QQSIRIMTIKHP (SEQ ID NO: 20), WRHPRLRCGNLL (SEQ ID NO:21), QKSRNRMTRTHP (SEQ ID NO: 22), SRARLFVVTYHK (SEQ ID NO: 23), HMISTMNAASRR (SEQ ID NO: 24), RSIVTFSLRQNR (SEQ ID NO: 25), RNTIRIRTIKHP (SEQ ID NO: 26) or RHSSTLRYRPLP (SEQ ID NO: 27).

Variants of the peptides according to the invention, the amino acid sequence of which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the amino acid sequences which correspond to the sequences in SEQ ID NOs: 1-27, preferably 1-22, more preferably 1-15, 16-17 or 18-22, more preferably 1-15 or 16-17, in particular 1-15, preferably differ in a maximum of 3 positions, more preferably in a maximum of 2 positions, in particular in a maximum of 1 position from one of the amino acid sequences, which correspond to the sequences in SEQ ID NOs: 1-27, preferably 1-22, more preferably 1-15, 16-17, or 18-22, more preferably 1-15 or 16-17, in particular 1-15. For example, if 1 position is different, either one of the amino acids of the respective sequence can be replaced or else the sequences match, but an amino acid within the amino acid chain or N- or C-terminal has been added or omitted. For example, a cysteine (C) could have been attached to the C- or N-terminal.

The term “variant”, as used herein, refers to variants of the peptide according to the invention which continue to have the functionality of the starting peptide but differ from the starting sequence by one or more sequence deviations, for example 1, 2 or 3 sequence deviations, for example a substitution, deletion or insertion. The sequence identity of such variants may be in the range of 80% based on the total length of the starting peptide, and may be at least 81, 82, 83, 84, 85, 86, 87, 88, 89, 90, 91, 92, 93, 94, 95, 96, 97, 98, 99 or 99.5%.

The identity of nucleic acid or amino acid sequences is determined by a sequence comparison. This sequence comparison is based on the BLAST algorithm, which is established and commonly used in the prior art (cf., for example, Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S.3389-3402) and in principle occurs in that similar sequences of nucleotides or amino acids in the nucleic acid or amino acid sequences are assigned to one another. A tabular assignment of the relevant positions is referred to as an alignment. A further algorithm available in the prior art is the FASTA algorithm. Sequence comparisons (alignments), in particular multiple sequence comparisons, are created using computer programs. Frequently used are for example the Clustal series (cf., for example, Chenna et al. (2003): Multiple sequence alignment with the Clustal series of programs. Nucleic Acid Research 31, 3497-3500), T-Coffee (cf., for example, Notredame et al. (2000): T-Coffee: A novel method for multiple sequence alignments. J. Mol. Biol. 302, 205-217) or programs based on these programs or algorithms are frequently used, for example. Also possible are sequence comparisons (alignments) using the computer program Vector NTIR Suite 10.3 (Invitrogen Corporation, 1600 Faraday Avenue, Carlsbad, California, USA) with the given standard parameters, the AlignX module of which is based on ClustalW for the sequence comparisons. Unless stated otherwise, the sequence identity indicated herein is determined using the BLAST algorithm.

Such a comparison also allows a conclusion to be drawn about the similarity of the compared sequences to one another. It is usually given in percent identity, i.e., the proportion of identical nucleotides or amino acid functional groups at the same positions or positions corresponding to one another in an alignment. In the case of amino acid sequences, the broader concept of homology takes conserved amino acid exchanges into account, i.e., amino acids having similar chemical activity, as these usually perform similar chemical activities within the peptide/protein. Therefore, the similarity of the compared sequences can also be indicated as percent homology or percent similarity. Identity and/or homology information can be provided regarding whole peptides, polypeptides or genes or only regarding individual regions. Homologous or identical regions of different nucleic acid or amino acid sequences are therefore defined by matches in the sequences. Such regions often have identical functions. They can be small and comprise only a few nucleotides or amino acids. Such small regions, however, often perform essential functions for the overall activity of the peptide/protein. It may therefore be expedient to relate sequence matches only to individual, optionally small, regions. Unless otherwise stated, however, identity or homology information in the present application relates to the total length of the respective nucleic acid or amino acid sequence indicated.

The peptide or protein concentration can be determined using known methods, for example the BCA method (bicinchoninic acid; 2,2′-bichinolyl-4,4′-dicarboxylic acid) or the Biuret method (A. G. Gornall C. S. Bardawill and M. M. David, J. Biol. Chem., 177 (1948), pp. 751-766). A person skilled in the art in the field of peptide and protein technology is aware of a variety of suitable methods for determining peptide or protein concentration that can be used within the scope of this invention.

Peptides according to the invention may have amino acid changes, in particular amino acid substitutions, insertions or deletions. Such peptides are, for example, further developed by targeted genetic alteration, i.e., by mutagenesis methods, and optimized for specific purposes or with regard to specific properties (for example with regard to their stability, binding etc.).

For example, targeted mutations such as substitutions, insertions or deletions can be introduced into the known molecules in order to alter certain properties, for example. For this purpose, in particular the surface charges and/or the isoelectric point of the molecules and thus their interactions with a surface can be altered. For example, the net charge of the peptides can be altered in order to thereby influence the substrate binding. Alternatively or additionally, one or more corresponding mutations can, for example, increase the stability or adsorption of the peptide. Advantageous properties of individual mutations, e.g., individual substitutions, can complement one another.

For the description of substitutions that relate to exactly one amino acid position (amino acid exchanges), the following convention is applied herein: first, the naturally present amino acid is referred to in the form of the internationally used single-letter code, followed by the associated sequence position and finally the inserted amino acid. Multiple exchanges within the same polypeptide chain are separated by slashes. In the case of insertions, additional amino acids are named according to the sequence position. In the case of deletions, the missing amino acid is replaced by a symbol, for example a star or a dash, or a Δ is indicated before the corresponding position. For example, P9T describes the substitution of proline at position 9 by threonine, P9TH describes the insertion of histidine following the amino acid threonine at position 9 and P9* or ΔP9 describes the deletion of proline at position 9. This nomenclature is known to a person skilled in the art in the field of enzyme technology.

Thus, the invention also comprises peptides characterized in that they are obtainable from a peptide as described above as a starting molecule, for example from a molecule having one of the amino acid sequences according to SEQ ID NOs: 1-27, preferably 1-22, more preferably 1-15, 16-17 or 18-22, even more preferably 1-15 or 16-17, in particular 1-15, on which, for example, one or more amino acid substitutions, including, inter alia, single or multiple conservative amino acid substitutions, have been carried out, with the resulting peptide having at least 80% sequence identity to one of the amino acid sequences according to SEQ ID NOs: 1-27, preferably 1-22, more preferably 1-15, 16-17 or 18-22, even more preferably 1-15 or 16-17, in particular 1-15.

The term “conservative amino acid substitution” means the exchange (substitution) of one amino acid residue for another amino acid residue, with this exchange not resulting in a change in the polarity or charge at the position of the exchanged amino acid, e.g., the exchange of one non-polar amino acid residue for another non-polar amino acid residue. Conservative amino acid substitutions within the scope of the invention include, for example: G=A=S, I=V=L=M, D=E, N=Q, K=R, Y=F, S=T, G=A=I=V=L=M=Y=F=W=P=S=T. However, it may be preferred for such exchanges not to have glycine or tyrosine as the target amino acid or, for example, also not to have an amino acid which has a low alpha-helix-forming potential.

In preferred embodiments, the peptide according to the invention may also be modified. Preferred modifications can be, for example, coupling of the peptide according to the invention with particular other molecules or chemical groups, for example organic (macro)molecules, e.g., via a covalent bond or a linker via a suitable amino acid of the chain and/or N- and/or C-terminally.

If the peptide according to the invention is coupled with at least one further (macro) molecule, it may also be referred to as a peptide derivative. The peptide according to the invention is then derivatized. In such embodiments, the peptide can act as an adhesion tag, which causes a molecule coupled thereto to bind to the desired surface. Such molecules may also be referred to as conjugates.

In some embodiments, the mentioned peptides according to the invention which may, for example for coupling purposes, N- and/or C-terminally have the amino acid cysteine, are coupled (functionally modified) with biotin, preferably at a suitable amino acid of the chain and/or N- and/or C-terminally.

It is also possible that the peptide is part of a protein or polypeptide. Such proteins and polypeptides can be produced recombinantly, for example, as fusion proteins. In such embodiments, the peptide according to the invention is either N- or C-terminal localized to provide the adhesion of the whole molecule to a surface. In such embodiments, too, the peptide thus acts as adhesion tag.

In various preferred embodiments, the peptide according to the invention may
(i) comprise or consist of one of the following amino acid sequences: RALQALRALQALEAL-C (SEQ ID NO: 28), RALRALRALEALEAL-C (SEQ ID NO: 29), RALRALRALQALQAL-C (SEQ ID NO: 30), RALRALRALQALEAL-C (SEQ ID NO: 31), RALRALQALEALEAL-C (SEQ ID NO: 32), RALFEALQALFRALEAL-C (SEQ ID NO: 33), RALRALEALQALEA-C (SEQ ID NO: 34), RALFEALFRALEALR-C (SEQ ID NO: 35), RALFEALFRALEAL-C (SEQ ID NO: 36), RALEALFRALEAL-C (SEQ ID NO: 37), RALRALFEALEAL-C (SEQ ID NO: 38), RALEALFRALQALEAL-C (SEQ ID NO: 39), RALEALWRALQALEAL-C (SEQ ID NO: 40), RALEALWRALEAL-C (SEQ ID NO: 41), RALARALARALAQALA-C (SEQ ID NO: 42), RSIVTFSLRQNAQLA-C (SEQ ID NO: 43), RSIVTFSLRQNSEQA-C (SEQ ID NO: 44), in particular one of SEQ ID NOs: 28-34, 39-40 or 42-44, in particular 28-32, 34 or 42; or
(ii) comprise or consist of an amino acid sequence which has at least 80%, preferably at least 81%, at least 82%, at least 83%, at least 84%, or at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% or in particular 100% sequence identity to one of the amino acid sequences specified in SEQ ID NOs: 28-44 (28, 29, 30, 31, 32, 33, 34, 35, 36, 37, 38, 39, 40, 41, 42, 43 or 44), preferably to one of SEQ ID NOs: 28-34, 39-40 or 42-44, in particular 28-32, 34 or 42.

Furthermore, it is possible for the peptide according to the invention to
(i) comprise or consist of one of the amino acid sequences: GLHTSATNLYLH-C (SEQ ID NO: 45), QHSIRLLTIKKP-C (SEQ ID NO: 46), QQSIRIMTIKHP-C (SEQ ID NO: 47), WRHPRLRCGNLL-C (SEQ ID NO: 48), or QKSRNRMTRTHP-C (SEQ ID NO: 49); or
(ii) comprise or consist of an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% or in particular 100% sequence identity to one of the sequences specified in SEQ ID NOs: 45-49 (45, 46, 47, 48 or 49).

In various embodiments, the peptide may comprise or consist of an amino acid sequence according to SEQ ID NOs: 50-54 (SRARLFVVTYHK-C (SEQ ID NO: 50), HMISTMNAASRR-C (SEQ ID NO: 51), RSIVTFSLRQNR-C (SEQ ID NO: 52), RNTIRIRTIKHP-C (SEQ ID NO: 53), RHSSTLRYRPLP-C (SEQ ID NO: 54)), or a variant thereof, which has at least 80%, preferably at least 85%, more preferably at least 90%, in particular at least 95% sequence identity thereto.

In certain embodiments, the peptide is a peptide suitable for adhesion to plastic surfaces.

In the context of this invention, “adhesion” is understood to mean an interaction between the peptide and a surface, thereby allowing the peptide to adhere to the surface. Thus, an “adhesive peptide” refers to a peptide that has the ability to interact with a particular surface and/or to adhere to a particular surface. The adhesive peptides described herein preferably have a 10-fold, more preferably a 20-fold, 50-fold or 100-fold greater adhesion to a given surface than any alternative peptide of comparable length which has not been developed for this purpose and does not meet the sequence specifications described herein.

The terms “synthetic” and “plastic” are to be used synonymously and typically refer to solids comprising polymers as basic building blocks, although other (chemical) groups and molecules may be added, for example.

The term “solid” in this context refers to any property of solid substances, i.e., it is to be distinguished from liquid or gaseous substances. Solids can be hard and inflexible, as well as ductile, bendable and flexible.

It is particularly preferred that the plastic surface is a plastic surface with low surface energy (low surface energy polymer (LSEP) surface). These surfaces are typically inert surfaces which had to be activated first for functionalization or for further modifications. Common methods here include, but are not limited to, plasma or corona treatment as well as treatment with primers, such as solvents or oxidizing agents. Such pretreatment methods can be circumvented or avoided by utilizing the peptides described herein because the peptides described also bind the untreated LSEP surfaces well.

To determine the surface energy of a plastic surface, a contact angle measurement can be carried out, for example, without being limited thereto. The measured contact angle indicates to what extent a liquid can wet the surface of a solid. It can be read from this to what extent this liquid, for example a varnish or an adhesive, is suitable for treating the surface being examined. For a person skilled in the art in this field, contact angle measurement is a common measurement method.

Preferably, the plastic surface comprises or consists of a material selected from the following: polyester (PES), polyethylene (PE), polypropylene (PP), polyurethane (PU), polystyrene (PS), polyvinyl chloride (PVC), polycarbonate (PC), polyamide (PA), polyphenylene ether, polyphenylene sulfide, polyoxymethylene (POM), polymethyl methacrylate (PMA), polyethylene terephthalate (PET), polybutylene terephthalate (PBT), polytetrafluoroethylene (PTFE), polyhydroxyalkanoate (PHA), polyhydroxybutyrate (PHB) polyimide (PI), polylactide (PLA), polyvinylidene fluoride (PVDF), polyether ketone (PEK etc.), copolymers and/or mixtures thereof and/or polymeric foams thereof, preferably polyester (PES), polyethylene (PE), polypropylene (PP), polystyrene (PS), copolymers and/or mixtures thereof and/or polymeric foams thereof, in particular polyethylene (PE), polypropylene (PP), polystyrene (PS) or copolymers and/or mixtures thereof.

In preferred embodiments, the plastic surface may represent one or more of the following parts: composite components, films, packaging, plastic particles and/or textiles, for example functional textiles.

Preferably, the peptide according to the invention demonstrates adsorption to the plastic surface of 0.25 to 3, for example 0.25 to 1 or 0.4 to 1, measured by means of the semi-quantitative method for adhesion measurement, which is described, for example, in WO 2014/072313 A1 or herein in Example 2.

Using the direct BCA method for adhesion measurement, the preferred adhesion values are in the range of 25 to 100, in particular 40 to 100. This method is described, for example, in Example 3.

Good adhesion to plastic surfaces can be achieved, for example, by a peptide having a pronounced helix structure (in particular α-helix structure) and/or a high arginine content, in particular at one of the termini.

Particularly preferred peptides according to the invention, for adhesion to plastic surfaces, preferably to LSEP surfaces, in particular to polyethylene, polypropylene, polystyrene, copolymers and/or mixtures thereof, comprise or consist of an amino acid sequence of 4 to 50 amino acids, preferably 10 to 24 amino acids, for example 11, 12, 13, 14, 15, 16, 17, 18, 19, 20, 21, 22, 23 or 24 amino acids, in particular 12 to 19 amino acids, for example 12 to 18 amino acids, where
(a) the amino acid sequence in N- to C-terminal orientation has the following sequence

(C)ₘX₁X₂X₃(X₄)ₙX₅(C)ₒ,

where
X₁ is a positively charged amino acid, preferably R or K, more preferably R,
X₂ and X₃ are uncharged amino acids, preferably selected from A, L, M, S, I and Q, more preferably from A, L, M and Q, in particular from A and L,
each X₄ independently of one another is any amino acid, preferably with the exception of P, more preferably with the exception of P and G;
X₅ is any positively charged or uncharged amino acid, preferably R or an uncharged amino acid, more preferably Q, A or L, in particular A or L,
m and o are 0 or 1, where m+o=0 or 1; and
n is an integer from 0 to 46, preferably from 6 to 20, for example 9, 10, 11, 12 or 13;
or
b) the amino acid sequence has at least 80%, preferably at least 81%, at least 82%, at least 83%, at least 84% or at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 16-22, preferably 16-17 or 18-22.

Particularly preferably, the peptide according to the invention, which exhibits good adhesion in particular with respect to polyethylene, has an amino acid sequence of 4 to 50 amino acids, preferably 10 to 24 amino acids, the amino acid sequence having at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identity to one of the amino acid sequences corresponding to one of the sequences in SEQ ID NOs: 1-5, 7 and 16-17 or 28-32, 34, 43 and 44, in particular 1-5 and 7 or 28-32 and 34.

Particularly preferred peptides according to the invention, which exhibit good adhesion in particular with respect to polypropylene, have an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identify to one of the amino acid sequences corresponding to one of the sequences in SEQ ID NOs. 1, 2, 4, 5, 7, 16 and 17 or 28, 29, 31, 32, 34, 43 and 44, in particular 1, 2, 4, 5 and 7 or 28, 29, 31, 32 and 34.

Particularly preferred peptides according to the invention, which exhibit good adhesion in particular with respect to polystyrene, have an amino acid sequence which has at least 80%, preferably at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5%, in particular 100% sequence identify to one of the amino acid sequences corresponding to the sequences in SEQ ID NOs. 1-5, 7 and 15-17 or 28-32, 34 and 42-44.

Methods for determining the adhesion of the peptides according to the invention are described in the examples, although the determination is not limited to these methods and the person skilled in the art in this field can apply all known and suitable methods for determining adhesion.

Furthermore, the peptides according to the invention may also be at least one subunit (module) of a polypeptide, wherein the polypeptide may comprise a multimer of the sequences described herein, for example 1 to 30 repeats, more preferably 2 to 15 repeats, in particular 2 to 10 repeats, for example 2, 3, 4, 5 or 6 repeats of the peptides according to the invention. The polypeptide may comprise or consist of such multimers. In various embodiments, the individual peptide subunits of the polypeptide (multimer) can be separated from one another by spacers, for example by spacers having a length of 1 to 10 amino acids, preferably 1 to 4 amino acids.

Thus, a further aspect of the invention is also a peptide or polypeptide (multimer) comprising one or more, preferably two or more, peptides according to the invention, as described herein. In this context, the term “polypeptide” refers in particular to such peptides comprising 100 or more amino acids.

The peptides according to the invention can be chemically produced using known methods of peptide synthesis, for example by solid phase synthesis according to Merrifield.

However, it is preferred to produce the peptide according to the invention using recombinant methods. According to the invention, these are understood to mean all genetic engineering or microbiological methods which are based on introducing the genes for the peptides of interest into a host organism that is suitable for production, and the host organism transcribing and translating the genes (amalgamated in the context of this invention as biotechnological methods). For example, the introduction of the genes of interest occurs via vectors, in particular expression vectors; but also via such vectors that cause the gene of interest to be able to be inserted into an existing genetic element in the host organism, such as the chromosome or other vectors. The functional unit composed of a gene and a promoter and possibly other genetic elements is typically referred to as an “expression cassette.” However, for this purpose the expression cassette does not necessarily also have to be present as a physical unit.

Particularly preferably, the peptides according to the invention are produced as polypeptides (multimers) and subsequently cleaved into the functional peptides. Very particularly preferred multimers have 2 to 10 peptide units (each according to the invention), which in each case are separated from one another by spacers having a length of 0 to 4 amino acids (for example 1, 2, 3 or 4 amino acids). These spacers can consist, for example, of the amino acids glycine (G), alanine (A) and serine (S). Alternatively, the spacers may be or comprise interfaces for specific proteases/peptidases, in particular endopeptidases, or form such an interface together with parts of the peptide according to the invention.

It is possible for a person skilled in the art to use methods which are currently generally known, for example chemical synthesis or polymerase chain reaction (PCR), in conjunction with molecular biology and/or protein-chemical standard methods, to produce the corresponding nucleic acids and even complete genes on the basis of known DNA and/or amino acid sequences and to then utilize these to synthesize peptides and polypeptides in suitable host cells.

In particularly preferred embodiments, the peptide is produced by means of biotechnological methods, as defined above.

In the context of the present invention, vectors are understood to mean elements which consist of nucleic acids and which contain a gene of interest as a characterizing nucleic acid range. Vectors allow establishment of this gene in a species or a cell line over multiple generations or cell divisions as a stable genetic element that is replicating itself independently of the rest of the genome. Vectors are, in particular when used in bacteria, special plasmids, i.e., circular genetic elements. In genetic engineering, a distinction is made between, on the one hand, such vectors that are used for storage, and thus, in a manner of speaking, also for genetic engineering work, the so-called cloning vectors, and, on the other hand, those vectors that fulfill the function of realizing the gene of interest in the host cell, i.e., of enabling the expression of the peptide in question. These vectors are referred to as expression vectors.

The nucleic acid encoding a peptide according to the invention or a multimer of such a peptide, which constitutes a further aspect of the invention, may be cloned into a vector, for example. The molecular biological dimension of the invention, therefore, lies in vectors having the genes for the corresponding peptides. These may include vectors, for example, which are derived from bacterial plasmids, from viruses, or from bacteriophages, or predominantly synthetic vectors or plasmids with elements from a wide variety of origins. Using the further genetic elements which are present in each case, vectors are able to become established as stable units in the host cells in question over several generations. In the context of the invention, it is immaterial whether the vectors are established extrachromosomally as independent units, or are integrated into a chromosome. Which of the numerous systems known from the prior art is selected depends on the individual case. For example, the achievable copy number, the available selection systems, among them primarily antibiotic resistance, or the culturability of the host cells that are capable of accepting the vectors may be deciding factors.

The vectors form suitable starting points for molecular biological and biochemical investigations of the gene in question or the associated peptide, for developments according to the invention, and lastly for amplifying and producing peptides according to the invention. The vectors represent further aspects of the present invention.

Cloning vectors are preferred embodiments of the present invention. In addition to storage, they are suitable for biological amplification or selection of the gene of interest for characterization of the gene in question, for example via the creation of a restriction map or via sequencing. Cloning vectors are therefore also preferred embodiments of the present invention, since they represent a transportable and storable form of the claimed DNA. They are also preferred starting points for molecular biological techniques that are not tied to cells, for example the polymerase chain reaction.

Expression vectors are chemically similar to the cloning vectors, but differ in those partial sequences which enable the expression vectors to replicate in the host organisms which are optimized for the production of peptides and to express the gene contained therein. Expression vectors, which themselves bear the genetic elements necessary for expression, are preferred embodiments. The expression is influenced, for example, by promoters which regulate the transcription of the gene. Thus, the expression may take place due to the natural promoter which is originally localized in front of this gene, but also after genetic fusion, both by a promoter of the host cell provided on the expression vector or also by a modified or completely different promoter of another organism.

Expression vectors which can be regulated via changes in the culture conditions, such as the cell density or specific factors, or by adding certain compounds, are preferred embodiments.

Embodiments of the present invention may also be cell-free expression systems in which the peptide biosynthesis is replicated in vitro. Such expression systems are likewise established in the prior art.

In vivo synthesis of a peptide according to the invention, i.e. by living cells, requires transfer of the associated gene into a host cell, what is known as the transformation thereof. In principle, all organisms, i.e. prokaryotes, eukaryotes, or Cyanophyta, are suitable as host cells. Preferred host cells are those which may be easily managed genetically, which concerns, for example, transformation using the expression vector and stable establishment thereof, for example unicellular fungi, such as, for example, yeasts or bacteria. In addition, preferred host cells are characterized by good microbiological and biotechnological manageability. This relates, for example, to ease of culturing, high growth rates, low demands on fermentation media, and good production and secretion rates for foreign peptides. It is often necessary to experimentally determine the optimal expression systems for the individual case from the large number of different systems that are available according to the prior art. Each peptide according to the invention may be obtained in this way from a variety of host organisms.

Such host cells are a further aspect of the present invention. Those host cells of which the activity thereof can be regulated due to genetic regulation elements which are provided on the expression vector, for example, but which may also be present in these cells from the outset, represent preferred embodiments. Expression in said cells may be induced, for example, by controlled addition of chemical compounds used as activators, by changing the cultivation conditions or when a particular cell density is reached. This allows very cost-effective production of the peptides of interest.

Prokaryotic or bacterial cells are preferred host cells. Compared to eukaryotes, bacteria are generally characterized by shorter generation times and lower demands on the culturing conditions. Inexpensive methods for obtaining peptides according to the invention may thus be established. In the case of gram-negative bacteria, such as E. coli, a large number of peptides are secreted into the periplasmatic space, i.e., into the compartment between the two membranes enclosing the cells. This can be advantageous for specific applications. In contrast, gram-positive bacteria, such as Bacilli or Actinomycetes or other representatives of the Actinomycetales, do not have an outer membrane, so that secreted peptides are immediately released into the culture medium surrounding the cells, from which, according to another preferred embodiment, the expressed peptides according to the invention may be directly purified.

A variant of this principle is represented by expression systems in which additional genes, for example those provided on other vectors, influence the production of peptides according to the invention. These may be modified gene products, or gene products which are to be purified together with the peptide according to the invention.

On account of the extensive experience with, for example, molecular biological methods and the culturability with coliform bacteria, these represent preferred embodiments of the present invention. Particularly preferred are those of the genera Escherichia coli, in particular non-pathogenic strains suitable for biotechnological production.

Representative members of these genera are the K12 derivatives and the B strains of Escherichia coli. Strains which can be derived therefrom by genetic and/or microbiological methods known per se, and can thus be regarded as their derivatives. These are of greatest importance for genetic and microbiological work and are preferably used to develop methods according to the invention. Such derivatives may be altered with regard to their requirements for culture conditions, for example via deletion or insertion mutagenesis, may have different or additional selection markers, or may express different or additional peptides. These may in particular be those derivatives which, in addition to the peptide produced according to the invention, express further peptides of economic interest.

Preferred microorganisms are also those characterized by having been obtained after transformation using one of the vectors described above. These may be, for example, cloning vectors that have been introduced for storage and/or modification in any bacterial strain. Such steps are widespread in the storage and development of genetic elements in question. Since the relevant genetic elements can be directly transferred from these microorganisms into gram-negative bacteria suitable for expression, the previous transformation products are also realizations of the relevant subject matter of the invention.

Eukaryotic cells may also be suitable for producing peptides according to the invention. Examples thereof are fungi such as Actinomycetes or yeasts such as Saccharomyces or Kluyveromyces. This may be particularly advantageous, for example, when, in the context of their synthesis, the peptides undergo specific modifications which allow such systems. This includes, for example, the binding of low-molecular compounds such as membrane anchors or oligosaccharides. In the context of this invention, this would be an example of a functionally modified peptide.

The host cells of the method according to the invention are cultured and fermented in a manner known per se, for example discontinuous or continuous systems. In the first case, a suitable culture medium is inoculated with the recombinant bacterial strains, and the product is harvested from the medium after a period of time that is to be experimentally determined. Continuous fermentations are characterized by reaching a steady state in which cells partially die but also regrow over a comparatively long period of time and at the same time product can be removed from the medium.

Fermentation methods are well known per se from the prior art, and represent the actual large-scale production step, followed by a suitable purification method.

All fermentation methods which are based on one of the above-mentioned methods for producing recombinant peptides represent correspondingly preferred embodiments of this subject matter of the invention.

In this regard, the conditions that are optimal in each case for the production methods used, for the host cells and/or the peptides to be produced, must be experimentally determined according to the knowledge of a person skilled in the art, based on the previously optimized culture conditions of the strains in question, for example with regard to fermentation volume, media composition, oxygen supply, or agitator speed.

Fermentation methods, which are characterized in that the fermentation is carried out via an inflow strategy, are also considered. Here, the media components consumed by the continuous cultivation are fed; this is also referred to as a feeding strategy. Significant increases both in the cell density and in the dry biomass, and/or primarily in the activity of the peptide of interest, may be achieved in this way.

Analogously, the fermentation may also be designed in such a way that undesirable metabolic products are filtered out, or neutralized by adding a buffer, or counterions which are appropriate in each case.

The produced peptide may be subsequently harvested from the fermentation medium. This fermentation method is preferred over product preparation from the dry mass, but requires the provision of suitable secretion markers and transport systems.

In the absence of secretion, it is necessary, in certain circumstances, to purify the peptide from the cell mass; various methods for this purpose are also known, such as precipitation by ammonium sulfate or ethanol, for example, or chromatographic purification, to the point of homogeneity, if necessary. However, the majority of the described technical methods should manage with an enriched, stabilized preparation.

All of the elements already described above may be combined into a method in order to produce peptides according to the invention. These methods for producing the peptides according to the invention represent further aspects of the present invention. Numerous possible combinations of method steps are conceivable for each peptide according to the invention. Optimal conditions could be determined experimentally for each specific individual case.

A further aspect of the present invention further are (isolated) nucleic acid sequences encoding a peptide according to the invention, or encoding a peptide comprising or consisting of an amino acid sequence according to one of SEQ ID NOs: 1-27, preferably 1-15 and/or 16-17 and/or 18-22, more preferably 1-15 and/or 16-17, in particular 1-15, or variants thereof which have at least 90%, preferably at least 91%, more preferably at least 92%, more preferably at least 93%, even more preferably at least 94%, even more preferably at least 95%, even more preferably at least 96%, even more preferably at least 97%, even more preferably at least 98%, even more preferably at least 99%, in particular at least 99.5% sequence identity to the sequences mentioned.

Furthermore, the invention relates to vectors, in particular cloning or expression vectors, comprising at least one (isolated) nucleic acid sequence according to the invention.

In a further aspect, the invention relates to a host cell, which is transformed with and/or contains at least one (isolated) nucleic acid sequence according to the invention or a vector according to the invention.

In a further aspect, the invention relates to a method for producing the peptides according to the invention.

In the aspects mentioned, the peptide according to the invention may also be a peptide or polypeptide according to the invention comprising one or more, preferably two or more peptides according to the invention, as described herein.

Furthermore, the invention relates to means comprising or consisting of at least one peptide according to the invention.

The means according to the invention is preferably (i) a coating agent, a varnish, a paint, an adhesion promoter or an adhesive, preferably a water-based adhesive, and/or (ii) a washing or cleaning agent.

In preferred embodiments, the means according to the invention are coating agents, varnishes, paints, adhesion promoters or adhesives, preferably water-based adhesives, preferably for plastic surfaces, in particular for LSEP plastic surfaces, such as polystyrene, polyethylene, polypropylene, polystyrene and/or copolymers or mixtures thereof, in particular polyethylene, polypropylene, polystyrene and/or copolymers and/or mixtures thereof.

The peptides according to the invention or the means according to the invention can be used, for example, as adhesion promoters between at least two adjoining layers of a multilayer composite or in the case of coated substrates between the coating and the substrate.

Multilayer composites are used for a wide variety of purposes, for example as packaging materials (in particular composite films) or self-adhesive articles (multilayer composite composed at least of one carrier and one adhesive layer). The multilayer composites may contain at least two, preferably from two to five, layers, wherein the individual layers may have a thickness of 0.01 to 5 mm, for example.

The individual layers are preferably plastic surfaces, in particular LSEP plastic surfaces, for example (LSEP) plastic films or (LSEP) polymer films.

The above-mentioned peptides or means can be used as an adhesion promoter between at least two adjoining layers of the multilayer composite. Preferably, at least one of the adjoining layers is a layer made of a natural or synthetic polymer. Possible polymers are, in particular, polycondensates such as polyesters, polyadducts such as polyurethanes, polyamides, polycarbonates, or polyphenylene ethers, or polyphenylene sulfides, or polymers, which are obtainable by radical or ionic polymerization of ethylenically unsaturated compounds (known as radical polymers for short). Such radical polymers preferably consist of at least 60 wt. %, most preferably at least 80 wt. %, of so-called principal monomers, selected from C1 to C20 alkyl(meth)acrylates, vinyl esters of carboxylic acids containing up to 20 C atoms, vinyl aromatics containing up to 20 C atoms, ethylenically unsaturated nitriles, vinyl halides, vinyl ethers of alcohols containing 1 to 10 C atoms, aliphatic hydrocarbons containing 2 to 8 C atoms, and one or two double bonds, in particular ethylene and propylene, and/or cyclic hydrocarbons, in particular styrene. The peptide according to the invention may be applied to one of the adjoining layers; alternatively, the peptide may be applied to both layers.

The peptide may be present as an aqueous solution. In such embodiments, the peptide content of the solution is preferably 0.00001 to 5 parts by weight of peptide to 100 parts by weight of water, for example 0.00001 to 5 wt. %, preferably 0.0001 to 1 wt. %, in particular 0.001 to 0.1 wt. %, in each case based on the total weight of the solution. For the uses according to the invention in coating agents, adhesion promoters, or adhesives for plastic surfaces, the solution is preferably further diluted to a concentration of 0.001 to 10000 μg/ml water, preferably 1 to 10000 μg/ml water, in particular 10 to 1000 μg/ml water. After the application, drying may therefore initially take place to remove the water. The two adjoining layers may subsequently be joined by conventional methods, such as lamination.

Substrates are coated for many different purposes. Reference is made in particular to decorative coatings or protective coatings (collectively, varnishes) or also adhesive coatings, it being possible for the adhesive as such to be applied to the substrate or adhered in the form of a self-adhesive article (label or adhesive tape), for example. The substrate or the substrate surface may consist of any material. Likewise, the coating or the substrate-side surface of the coating may consist of any material. The substrate surface or the coating, or the substrate-side surface of the coating, preferably consists of plastic, in particular of polymers or copolymers.

Possible polymers are, in particular, polycondensates such as polyesters, polyadducts such as polyurethanes, polyamides, polycarbonates, or polyphenylene ethers, or polyphenylene sulfides, or polymers, which are obtainable by radical or ionic polymerization of ethylenically unsaturated compounds (known as radical polymers for short). Polymers or copolymers of ethylene, propylene and/or styrene are particularly preferred. Regarding the structure of radical polymers and their content of principal monomers, the above applies.

The quantity of the peptide necessary for adhesion promotion corresponds to the quantity stated above. The peptide may be applied to the substrate surface, to the substrate-side surface of the coating, or to both. The peptide is preferably present as an aqueous solution; the content of the solution is as indicated above. After the application, drying may therefore initially take place to remove the water.

The coating may be applied to the substrate according to conventional methods, and films or multilayer composites may be laminated on, for example. In particular, the coating may be produced by applying a polymer dispersion, a polymer solution, or a solvent-free polymer to the substrate-side surface provided with the adhesion promoter, and subsequent filming and/or thermal or photochemical curing. For this purpose, the polymer is present in particular in the form of an aqueous dispersion or solution, and is particularly preferably an aqueous dispersion of an emulsion polymer, preferably one of the radical polymers set forth above. After the polymer is applied, drying optionally takes place.

The multilayer composites and coated substrates thus preferably have significantly increased strength. Due to the use of the peptide according to the invention as an adhesion promoter, the coating preferably adheres more strongly to the substrate, and the individual layers of the multilayer composite adhere better to one another.

In one very particularly preferred embodiment of the invention, the substrate is a plastic. In principle, this may be any plastic. Examples comprise plastics made of polyester, polyethylene (PE), polypropylene (PP), polyurethane (PU), polystyrene (PS), polyvinyl chloride (PVC), polycarbonate (PC), polyamide (PA), polyphenyl ether, polyphenylene sulfide, polyoxymethylene (POM), polymethyl methacrylate (PMA), polyethylene terephthalate (PET), polybutylene terephthalate (PBT), polytetrafluoroethylene (PTFE), polyhydroxyalkanoate (PHA), polyhydroxybutyrate (PHB) polyimide (PI), polylactide (PLA), polyvinylidene fluoride (PVDF), polyetherketone (PEK etc.), copolymers thereof and/or polymeric foams thereof, preferably polyester (PES), polyethylene (PE), polypropylene (PP), polystyrene (PS), copolymers thereof and/or polymeric foams thereof, in particular polyethylene (PE), polypropylene (PP), polystyrene (PS), copolymers thereof and/or polymeric foams thereof. The plastics may be present in any form, but preferably are plastic films, plastic strips, or plastic packaging. Textiles made of plastic or a high plastic content (at least 10%, more preferably at least 20%, even more preferably at least 30%, even more preferably at least 40%, even more preferably at least 50%, in particular 60% plastic content, for example 65%) are also comprised. The plastic may also be a composite material having a plastic surface.

The plastic surfaces are coated with the peptides according to the invention or the means according to the invention as adhesion promoters. This may be carried out using aqueous solutions of the peptides. Details of the coating have already been given above.

The coating may in particular be typical paints or paint systems for coating plastic surfaces. These paints or paint systems may be paints which cure thermally, photochemically, or by other mechanisms.

Typical paints for coating plastic surfaces comprise at least one binder as well as crosslinkable components. The crosslinkable components may be crosslinkers which are used in addition to a binder, or may be crosslinkable groups which are joined to the binder. Of course, the binder may also have crosslinkable groups, and in addition a crosslinker may be used. Various possible combinations are conceivable. For example, the binder and the crosslinker may be used separately. The binder then includes reactive functional groups which are able to react with complementary, reactive functional groups in the crosslinkers. Alternatively, the binders may be self-crosslinking binders which include reactive functional groups that are able to take part in crosslinking reactions with groups of their type (“with themselves”) or with complementary, reactive functional groups on the same polymer. It is also possible that only the crosslinkers react with one another.

Examples of suitable binders comprise (meth)acrylate (co)polymers, partially saponified polyvinyl esters, polyesters, alkyd resins, polylactones, polycarbonates, polyethers, epoxy resin-amine adducts, polyureas, polyamides, polyimides, or polyurethanes. Of course, mixtures of various polymers may also be used, provided that no undesirable effects result from the mixture. The crosslinking components may have thermally crosslinking groups or photochemically crosslinking groups. Suitable thermal crosslinkers are, for example, crosslinkers based on epoxides, melamine, or blocked isocyanates. Suitable crosslinkers for photochemical crosslinking are in particular compounds containing multiple ethylenically unsaturated groups, in particular di- or polyfunctional acrylates. By means of the peptides according to the invention or the means according to the invention, the adhesion of the coating to the substrate is advantageously improved. In addition, improved resistance against infiltration of the paint layer is preferably achieved in corrosion protection tests.

During bonding, two plastic surfaces, where preferably at least one, but for example also both plastic surfaces have been treated with the peptide according to the invention, are preferably brought into contact with one another, or a plastic surface treated with the peptide is brought into contact with another surface.

The surface treatment relates in particular to composite components or other (plastic) surfaces which are pretreated with the peptide according to the invention or the means according to the invention prior to coating, varnishing, painting or bonding.

In other preferred embodiments, the means according to the invention is a washing or cleaning agent, in particular for use in textiles made of plastic or having a plastic content (preferably at least 10%, more preferably at least 20%, even more preferably at least 30%, even more preferably at least 40%, even more preferably at least 50%, in particular 60% plastic content, for example 65%), for example functional clothing. Here, the textiles are preferably made of plastic or plastic mixtures, for example polyester, polyethylene, polypropylene, polystyrene and/or copolymers and/or mixtures thereof.

A further aspect of the invention relates to a method for bonding, coating, varnishing and/or surface treatment of plastic surfaces, preferably LSEP surfaces, wherein at least one peptide according to the invention or a means according to the invention is used in at least one method step.

A further aspect of the present invention relates to the use of the peptide according to the invention or of the peptide or polypeptide (multimer) according to the invention comprising one or more, preferably two or more peptides according to the invention, or the means described herein for binding/adhesion to plastic surfaces, in particular LSEP surfaces. The use may also comprise the use of the peptides described herein as components of larger molecules to mediate their binding to a surface.

A further aspect of the present invention is the use of a peptide as described herein or corresponding conjugates, multimers or means for adhesion to plastic surfaces, preferably LSEP surfaces, more preferably polyester, polyethylene, polypropylene, polystyrene or copolymers and/or mixtures thereof, in particular polyethylene, polypropylene, polystyrene or copolymers and/or mixtures thereof, where the peptide or polypeptide has or consists of at least one amino acid sequence according to one of SEQ ID NOs. 1-27, for example 1-15 or 16-17 or 18-22 or 23-27, preferably 1-17 or 18-22, more preferably 1-17, in particular 1-15, or is a variant thereof that has at least 80%, preferably at least 81%, at least 82%, at least 83%, at least 84%, or at least 85%, more preferably at least 86%, even more preferably at least 87%, even more preferably at least 88%, even more preferably at least 89%, even more preferably at least 90%, even more preferably at least 91%, even more preferably at least 92%, even more preferably at least 93%, even more preferably at least 94%, even more preferably at least 95%, even more preferably at least 96%, even more preferably at least 97%, even more preferably at least 98%, even more preferably at least 99%, even more preferably at least 99.5% sequence identity to one of the specified sequences.

In preferred embodiments, the peptide or means according to the invention is used for bonding, coating, varnishing and/or surface treatment of plastic surfaces, preferably LSEP surfaces, as well as of multilayer composites or coated substrates, or for degradation of plastic. Preferably, the peptide according to the invention is used as a component of coating agents, varnishes, paints, adhesion promoters or adhesives for plastic surfaces as well as in multilayer composites or coated substrates, or the means according to the invention is used as coating agent, varnish, paint, adhesion promoter or adhesive for plastic surfaces as well as in multilayer composites or coated substrates.

In various other embodiments, the peptide or means according to the invention may be used for the degradation of plastics, preferably LSEP microplastics and/or for cleaning or treating textiles made of plastic or having a plastic content (preferably at least 10%, more preferably at least 20%, even more preferably at least 30%, even more preferably at least 40%, even more preferably at least 50%, in particular 60% plastic content, for example 65%).

In various embodiments, the peptide according to the invention may be functionally modified/derivatized/conjugated in these uses as described above and/or it is contained in a peptide or polypeptide (multimer) comprising one or more, preferably two or more, of the peptide according to the invention.

Preferably, the peptide according to the invention or the means according to the invention is used at a pH of from 2 to 12, preferably from 5 to 9, for example from 7 to 9. This may refer, for example, to the pH of the finished means, to the ready-to-use composition or, for example, to the pH of the washing liquor in which the means or peptide is used.

### EXAMPLES

### Example 1: Peptide Sequences

TABLE 1
Peptide names and associated amino acid sequences.

| | Peptide | |
|---|---|---|
| No. | name | Amino Acid Sequence |
| 1 | BA1-A06 | GLHTSATNLYLH (SEQ ID NO: 18) |
| 2 | D2 | QHSIRLLTIKKP (SEQ ID NO: 19) |
| 3 | D4 | QQSIRIMTIKHP (SEQ ID NO: 20) |
| 4 | P3 | WRHPRLRCGNLL (SEQ ID NO: 21) |
| 5 | P6 | SRARLFVVTYHK (SEQ ID NO: 23) |
| 6 | P7 | HMISTMNAASRR (SEQ ID NO: 24) |
| 7 | P8 | RSIVTFSLRQNR (SEQ ID NO: 25) |
| 8 | P8_S2 | RSIVTFSLRQNAQLA (SEQ ID NO: 16) |
| 9 | P8_S8 | RSIVTFSLRQNSEQA (SEQ ID NO: 17) |
| 10 | P13 | RNTIRIRTIKHP (SEQ ID NO: 26) |
| 11 | P14 | QKSRNRMTRTHP (SEQ ID NO: 22) |
| 12 | P15 | RHSSTLRYRPLP (SEQ ID NO: 27) |
| 13 | RAL-M | RALQALRALQALEAL (SEQ ID NO: 1) |
| 14 | RAL2E | RALRALRALEALEAL (SEQ ID NO: 2) |
| 15 | RAL2Q | RALRALRALQALQAL (SEQ ID NO: 3) |
| 16 | RALQE | RALRALRALQALEAL (SEQ ID NO: 4) |
| 17 | RALQ2E | RALRALQALEALEAL (SEQ ID NO: 5) |
| 18 | RAL2FQ | RALFEALQALFRALEAL (SEQ ID NO: 6) |
| 19 | RALEQE | RALRALEALQALEA (SEQ ID NO: 7) |
| 20 | RAL2FR | RALFEALFRALEALR (SEQ ID NO: 8) |
| 21 | RAL2F | RALFEALFRALEAL (SEQ ID NO: 9) |
| 22 | RALF | RALEALFRALEAL (SEQ ID NO: 10) |
| 23 | RALF_sym | RALRALFEALEAL (SEQ ID NO: 11) |
| 24 | RALF_Q | RALEALFRALQALEAL (SEQ ID NO: 12) |
| 25 | RALFQ_W | RALEALWRALQALEAL (SEQ ID NO: 13) |
| 26 | RALF_W | RALEALWRALEAL (SEQ ID NO: 14) |
| 27 | RALA4Q | RALARALARALAQALA (SEQ ID NO: 15) |

The peptides originate in part from E. coli display database screenings (e.g., P3, P6, P15, P8C); other peptides are either further developments thereof or pure designer peptides (for example RAL peptides or variants thereof). The peptide can be present as listed or, for example, can be supplemented at the N- and/or C-terminal by another amino acid such as cysteine. Furthermore, the peptide can, for example, be coupled to another peptide or molecule such as biotin (functionalized peptide). For example, P3 represents the peptide sequence without cysteine, P3C or 1-C represents the peptide sequence with C-terminal cysteine, P3-Bt represents the peptide sequence with C-terminal biotin. Furthermore, the peptide can also occur as a subunit (module) in a polypeptide.

TABLE 2
Charge of the peptide regions.

| No. | Amino Acid Sequence |
|---|---|
| 8 | RSIVTFSLRQNAQLA (SEQ ID NO: 16) |
| 9 | RSIVTFSLRQNSEQA (SEQ ID NO: 17) |
| 13 | RALQALRALQALEAL (SEQ ID NO: 1) |
| 14 | RALRALRALEALEAL (SEQ ID NO: 2) |
| 15 | RALRALRALQALQAL (SEQ ID NO: 3) |
| 16 | RALRALRALQALEAL (SEQ ID NO: 4) |
| 17 | RALRALQALEALEAL (SEQ ID NO: 5) |
| 19 | RALRALEALQALEAL (SEQ ID NO: 7) |

With reference to Table 2, underlined amino acids denote parts of the peptide (tripeptides) having a positive charge, amino acids in bold denote regions of the peptide which are uncharged, and amino acids highlighted in gray denote parts of the peptide (tripeptides) having a negative charge.

### Example 2: Testing the Adhesion of the Synthesized Peptides With Regard to Their Substrate Specificity on Polyethylene (PE), Polypropylene (PP) and Polystyrene (PS)

Here, the adhesion of non-functionalized peptides (Nos. 1-4, 10, 11) and peptides having the amino acid cysteine at the C-terminus (Nos. 5-C to 9-C, 13-C to 17-C, 19-C, 27-C) to LSEP surfaces such as polyethylene, polypropylene and polystyrene was examined; AI-S1 (VPSSGPQDTRTT) (SEQ ID NO: 55) and MS-S1 (ATIHDAFYSAPE) (SEQ ID NO: 56) are aluminum- or steel-binding peptides, respectively, which served as negative controls. These peptides are known from Zuo R., Örnek, D., Wood, T. K. (2005) Aluminum and mild steel-binding peptides from phage display, Appl. Microbiol. Biotechnol., 68: 505-509.

For the examination, the peptides were dissolved in 1×PBS buffer pH 7.3 (without NaCl and KCl) to a concentration of 2 mg/mL. 2×10 μL each were dropped onto an LSEP plate (40 μg). The “peptide spots” were dried at 30° C. for 30 min, then washed for 1 h in 1 L 1×PBS buffer with gentle shaking and stained for 30 s in Coomassie staining solution. Decolorizing was carried out with 1×PBS buffer pH 7.3 (without NaCl and KCl). Depending on the intensity of the bound color on the peptide spots, an optical semi-quantitative evaluation was performed.

### Semi-Quantitative Evaluation of the Adhesion

| | |
|---|---|
| 0 | No adhesion |
| 0.5 | Low adhesion |
| 1 | Average adhesion |
| 2 | Good adhesion |
| 3 | Very good adhesion |

TABLE 3
Examination of the adhesion of peptides on various LSEP plates.

| | | Material | | |
|---|---|---|---|---|
| Peptide | | Polyethylene | Polypropylene | Polystyrene |
| No. | Name | (PE) | (PP) | (PS) |
| | LSEP binding peptide | | | |
| 1 | BA1-A06 | 0.5 | 0.5 | 1 |
| 2 | D2 | 0.0 | 0.0 | 0.5 |
| 3 | D4 | 0.5 | 0.5 | 1 |
| 4 | P3 | 0.5 | 0.5 | 1 |
| 5-C | P6 | 2 | 3 | 3 |
| 6-C | P7 | 0.5 | 0.5 | 1 |
| 7-C | P8 | 1 | 2 | 2 |
| 8-C | P8_S2 | 3 | 3 | 3 |
| 9-C | P8_S8 | 2 | 2 | 3 |
| 10 | P13 | 0.5 | 0.5 | 0.5 |
| 11 | P14 | 0.5 | 0 | 0.5 |
| 12 | P15 | 0.5 | 0 | 0.5 |
| 13-C | RAL-M | 2 | 3 | 3 |
| 14-C | RAL2E | 3 | 3 | 3 |
| 15-C | RAL2Q | 3 | 1 | 3 |
| 16-C | RALQE | 3 | 3 | 3 |
| 17-C | RALQ2E | 2 | 3 | 3 |
| 19-C | RALEQE | 2 | 3 | 3 |
| 27-C | RALA4Q | 1 | 1 | 2 |
| | Negative controls | | | |
| | AI-S1 | 0 | 0 | 0 |
| | MS-S1 | 0 | 0 | 0 |

Good to very good adhesion to polyethylene was found for peptides 5-C, 8-C, 9-C, 13-C, 14-C, 15-C, 16-C, 17-C, and 19C. To polypropylene, peptides 5-C, 7-C, 8-C, 9-C, 13-C, 14-C, 16-C, 17-C, and 19-C bind good to very good, and to polystyrene, peptides 5-C, 7-C, 8-C, 9-C, 13-C, 14-C, 15-C, 16-C, 17-C, 19-C, and 27-C bind good to very good.

### Example 3: Testing the Adhesion of the Synthesized Peptides With Regard to Their Substrate Specificity on Polypropylene (PP)

### Direct Measurement Method of Adhesion Measurement With BCA

50 μL 0.1 mg/mL peptide solution was added to a 96-well plate made of polypropylene. The “peptide spots” were dried at 30° C. for 16 h in the incubator. Then 0.2 mL each of aqua dest. were added and incubated for 10, 30, 60 and 90 min at RT, shaking at 300 rpm. 100 μL of the supernatant was transferred and 50 μL of BCA reagent was added and incubated for 30 min at 60° C. The absorption was measured at 562 nm. An adhesion point after an infinite adhesion time can be read from the measured adhesion values.

### Evaluation of the Adhesion in %

| | |
|---|---|
| <20 | No adhesion |
| 21 bis 40 | Low adhesion |
| 41 bis 60 | Average adhesion |
| 61 bis 80 | Good adhesion |
| >80 | Very good adhesion |

TABLE 4
Examination of the adhesion of peptides, which
C-terminally have a cysteine, on polypropylene.

| | | Material |
|---|---|---|
| | | Adhesion % to |
| Peptide | | polypropylene |
| No. | Name | (PP) |

LSEP binding peptide

| | | |
|---|---|---|
| 7-C | P8 | 21 |
| 9-C | P8_S8 | 62 |
| 13-C | RAL-M | 45 |
| 14-C | RAL2E | 43 |
| 17-C | RALQ2E | 46 |
| 18-C | RAL2FQ | 97 |
| 19-C | RALEQE | 50 |
| 20-C | RAL2FR | 54 |
| 21-C | RAL2F | 56 |
| 22-C | RALF | 28 |
| 23-C | RALF_sym | 23 |
| 24-C | RALF_Q | 83 |
| 25-C | RALFQ_W | 99 |
| 26-C | RALF_W | 25 |

Negative control

| | |
|---|---|
| MS-S1 | 5 |

Peptides 9-C, 18-C, 24-C and 25-C showed good to very good adhesion to polypropylene. Average adhesions were determined for 13-C, 14-C, 17-C, 19-C, 20-C and 21-C.

1. A peptide comprising or consisting of an amino acid sequence of 4 to 50 amino acids, preferably 10 to 24 amino acids, where
(a) the amino acid sequence in N- to C-terminal orientation has the following sequence
(C)ₘX₁X₂X₃(X₄)ₙX₅(C)ₒ,
where
X₁ is a positively charged amino acid, preferably R or K, more preferably R,
X₂ and X₃ are uncharged amino acids, preferably selected from A, L, M and Q, more preferably from A and L,
each X₄ independently of one another is any amino acid, preferably with the exception of P, more preferably with the exception of P and G;
X₅ is any positively charged or uncharged amino acid, preferably R or an uncharged amino acid, more preferably Q, A or L, in particular A or L,
m and o are 0 or 1, where m+o=0 or 1; and
n is an integer from 0 to 46, preferably from 6 to 20;
or
b) the amino acid sequence has at least 80%, preferably at least 85%, more preferably at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5% or 100% sequence identity to one of the amino acid sequences specified in SEQ ID NOs: 16-17 or 18-22.

2. The peptide according to claim 1, where
(i) the peptide has a total charge of 0 to +4, preferably 0 to +2; and/or
(ii) the N-terminus comprising the first 3-4 amino acids has a net positive charge, and/or
(iii) the C-terminus comprising the last 3-6 amino acids has a net negative or neutral charge and preferably comprises at least one negatively charged amino acid, in particular E, and/or
(iv) the peptide contains no P and preferably also no G and more preferably also no Y.

3. The peptide according to claim 1, where the sequence X₁X₂X₃ is RAL or RLA, preferably RAL.

4. The peptide according to claim 1, where
(iii) (X₄)ₙX₅ comprises at least one sequence X₆X₇X₈, where X₆ is a charged or uncharged amino acid, preferably R, K, E, or Q, and X₇ and X₈ independently of one another are uncharged amino acids with the exception of P and G, preferably A, L, or M, more preferably A or L;
(iv) (X₄)ₙ comprises at least one aromatic amino acid, preferably W or F.

5. The peptide according to claim 4, where
(i) (X₄)ₙ comprises at least one sequence X₆X₇X₈, where X₆X₇X₈ is RAL or RLA, preferably RAL, and where said sequence is preferably localized in the N-terminal amino acids of positions 4-7; and/or
(ii) (X₄)ₙX₅ comprises at least one sequence X₆X₇X₈, where X₆X₇X₈ is EAL or ELA, preferably EAL, and where said sequence is preferably not localized in the N-terminal amino acids of positions 1-6; and/or
(iii) (X₄)ₙX₅ comprises at least one sequence X₆X₇X₈, where X₆X₇X₈ is QAL or QLA, preferably QAL.

6. The peptide according to claim 5, where the peptide comprises the sequence X₁X₂X₃ RAL and (X₄)ₙX₅ comprises at least one of QAL and EAL, preferably both.

7. The peptide according to claim 6, where the peptide additionally comprises at least one further sequence RAL.

8. The peptide according to claim 6, where the peptide contains two sequences RAL and at least one sequence each of EAL and QAL.

9. The peptide according to claim 6, where the peptide contains at least one W or F, preferably exactly one W or F.

10. The peptide according to claim 1, where the peptide consists of at least 60%, preferably at least 65%, more preferably at least 70%, in particular at least 75% amino acids having a high alpha-helix-forming potential, with said amino acids preferably being selected from E, A, L, M, Q, K, R, F, I, H, W and D, more preferably E, A, L, M, Q, K, R, F, I and H; even more preferably E, A, L, M, Q, K, R and F.

11. The peptide according to claim 1, where the peptide has the amino acid sequence according to one of SEQ ID NOs: 1-15, as well as variants thereof, which have at least 80%, preferably at least 90%, sequence identity to the specified sequence, with preferably the RAL motif, and preferably also the EAL and/or QAL motif, if present, being invariable.

12. The peptide according to claim 1, where the peptide is a peptide for adhesion to a plastic surface, preferably to an LSEP surface, in particular to a surface made of polyethylene (PE), polypropylene (PP) or polystyrene (PS) or copolymers or mixtures thereof.

13. A peptide or polypeptide (multimer) comprising two or more of the peptides according claim 1.

14. A nucleic acid sequence encoding for a peptide according to claim 1, preferably for a peptide comprising or consisting of an amino acid sequence according to one of the SEQ ID NOs: 1-15, 16-17 or 18-22.

15. A means comprising at least one peptide according to claim 1.

16. A use of a peptide for adhesion to plastic surfaces, preferably LSEP surfaces, where the peptide has at least one amino acid sequence according to a sequence of SEQ ID NOs: 1-27, preferably of SEQ ID NOs: 1-15, 16-17 or 18-22, more preferably 1-15 or 16-17, in particular 1-15, or a variant thereof, which has at least 80%, preferably at least 90%, sequence identity to one of the specified sequences, with the peptide, in preferred embodiments, being a peptide according to claim 1.
